(19) United States
(12) Patent Application Publication
(10) Pub. No.: US 2001/0039539 A1
(43) Pub. Date: Nov. 8, 2001

(54) Database assisted experimental procedure

(76) Inventors: Adam Sartiel, Zur Igal (IL); Lior Ma'Ayan, Ramat Hasharon (IL)

Correspondence Address: BIRCH STEWART KOLASCH & BIRCH, PO BOX 747, FALLS CHURCH, VA, 22040-0747, US

(21) Appl. No.: 09/732,941

(22) Filed: Dec. 11, 2000

(30) Foreign Application Priority Data
Dec. 12, 1999 (IL) 133475
Feb. 17, 2000 (IL) 134587
Mar. 31, 2000 (IL) 135393

(51) Int. Cl. G06F 7/00

(52) U.S. Cl. 707/1

(57) ABSTRACT

A method for performing an experiment is provided, which method comprises providing or selecting experiment tools, performing the experiment with the aid of said tools and analyzing the results. A data-containing site on a computer network is communicated through the network to extract therefrom experiment-specific data This data is utilized for either designing or selecting the tools, defining parameters or manner of performing the experiment, predicting the outcome of one or more subsequent steps of the experiment and/or analyzing the results or data obtained in the experiment.

### FIELD AND BACKGROUND OF THE INVENTION

The present invention concerns a method of performance of an experimental procedure. By preferred embodiments of the invention, the experimental procedure is carried out, at least partially in an apparatus the operation of which is controlled by a control processor.

Experimental procedures are aimed and designed to increase the available scientific data. For example, in the human genome project which involves sequencing through automatic high through-put biological or biochemical experimental procedures, generates vast sequence data that is eventually used to gain information on the human genome. The sequencing endeavor is typically an open and at procedures in that the result flows continuously into databases and is used to generate data which is then made available for research.

### GENERAL DESCRIPTION OF THE INVENTION

In the following the term “experimental procedure” and “experiment” will be used interchangeably to relate to a procedure or process intended to study a scientific phenomena, collect scientific data or a procedure intended to design probes, media or other experiment tools for carrying out a specific type of an experiment (typically for the purpose of optimizing such tools to increase accuracy of experiment, improve ability to analyze results or data from the such an experiment etc.). The present invention provides a novel concept in carrying out experimental procedures. In accordance with the invention a database containing scientific data (data obtained from scientific results or from analysis of such results) is created and placed at a site on a computer network, typically the Internet, in performance of an experimental procedure, an experimenter, which may be a human individual or a processor-control apparatus, communicates with the database over the computer network and then extracts data relevant to the performance of the experimental procedure, in the following the term “experiment-specific data” will be used to denote data which may be employed in a specific experiment for either designing or selecting experiment tools to be employed in the experiment defining parameters of the experiments or the manner in which it is performed (e.g. sequence of steps), or employed in analyzing the results or data which is obtained in the experiment.

The term “biological or biochemical experimental procedure” refers to different kinds of procedures aimed in analyzing biological samples, experimental procedures aimed in gaining knowledge on a certain biologically significant fact or principle, an experiment designed to study function of biochemical entities (macromolecules and other molecules) having a biological activity or being derived from a biological sample, a diagnostic or screening assay, etc. In general, a biological or biochemical experimental procedure includes any experimental procedure which may yield a result having a meaning or significance in the field of life sciences or which may yield an outcome which may be relevant for the purpose of other experimental procedures or subsequent steps of an experimental procedure. (An “outcome” may, for example, be a certain biochemical reaction result, a conditioned medium, etc., which while not being diagnosed or assayed per se, is used as a starting, material, as a reagent, etc., for other experimental procedures or subsequent steps of an experimental procedure).

The term “experiment tools” will be used to denote any physical (as opposed to “virtual” component used in an experiment such as chemicals, reagents, media, reaction vessels or substrates, etc.

In accordance with one of its aspects, the present invention provides a method for performing an experiment comprising:

(a) providing or selecting one or more experiment tools,

(b) performing said experiment utilizing said tools, and

(c) analyzing results;

said method being characterized in that it comprises communicating through a computer network with a data-containing site of the network to extract therefrom experiment-specific data and utilizing said data for either one or more of (i) designing or selecting said tools, (ii) defining parameters or manner of performing the experiment (iii) predicting outcome of one or more subsequent steps of the experiment, and (iv) analyzing the results or data obtained in the experiment.

In accordance with one preferred embodiment of the invention, the experimental procedure is a biological or biochemical experimental procedure. Such an experimental procedure may be intended to analyze or utilize biological macromolecules in which case said data is a data on composition, chemical properties, physical properties, biological properties or function, distribution thereof in different biological tissues, and data on closely related macromolecules (e.g. data on homologues). Preferably, said macromolecules are nucleic acid sequences or polypeptides. The term “composition” includes, for example, the type of building blocks, subunits, etc. which make up the macromolecule. The term “chemical properties” refers to its reactivity, the PK of the macromolecule, its ability to react with other macromolecules (e.g. hybridization in the case of polynucleotides) and the conditions under which such reaction takes place, etc. The term “physical properties” refers to properties such as size, three-dimensional structure, molecular weight, nature of its binding site, etc. The term “biological properties” or “function” refers to the biological role within the cell or the body, etc. The term “distribution” refers to the pattern of its appearance in different cells or tissue. The term “closely related macromolecules” refers to homologies; e.g. macromolecules with a similar sequence, etc,

By another of its aspects the invention provides a method for assembling experiment tools for use in an experiment, comprising annexing to or associating with id tools means or an instruction set for communicating, over a computer network, with a data-containing site and extracting from sad site data relevant to the experiment, the extracted data being employed to (i) define parameters or manner of performing the experiment, or (ii) analyze results or data obtained in the experiment.

The term “annexing to” or “associating with” means to denote either physically packing together or providing such means for use with a specific set of experiment tools. The term “instruction set” denotes instructions for communicating and extracting experiment-specific data which may be in a printed or an electronic form. Such means typically comprise a computer readable medium which carries a software which can be loaded into a computer or into a processor of an apparatus that executes the experiment. By virtue of such software, said computer or apparatus can communicate with said site, extract the experiment-specific data and process such data for use in the experiment,

In accordance with a preferred embodiment of this aspect, said means comprise a computer readable medium carrying software loadable into a computer or into a processor of an apparatus to execute the experiment, whereby said computer or apparatus can communicate with said site, extract said experiment-specific data and process it for use in said experiment.

In accordance with another embodiment of this aspect, said instruction set comprise the address of site containing said database as well as, at times, a code permitting extraction of experiment-specific data therefrom.

In accordance with another aspect the invention provides a method comprising:

(a) collecting scientific data

(b) assembling said data into a database in a manner permitting extraction therefrom of experiment-specific data and utilization of said data for either (i) design of tools for carrying out a specified experiment, (ii) define parameters for carrying out a specified experiment or (iii) analyze results or data obtained in an experiment;

(c) loading the database onto a site of a computer network in a manner permitting users of the network to communicate therewith and to exact therefrom the experiment-specific data for utilizing the extracted data in their experiment in a manner as defined in step (b).

By a still Her aspect the invention provides a method for assisting an experiment performer in performing an experimental procedure, comprising the steps of:

(a) providing a user accessible site on a computer network in a manner permitting users of the network to communicate and exchange data therewith, the site holding a database of data and software permitting to search the database and extract experiment-specific data therefrom, said data being data that may be used by the experimenter in designing an experimental procedure or analyze results or data obtained in such a procedure; and

(b) permitting the experimenter to communicate with said site through the computer network and in response to data transmitted from the experimenter to said site defining particulars of the experiment, transmitting to the experimenter said experiment-specific data or said data in combination with software, for (i) designing or selecting experiment tools for carrying out the experimental procedure, (ii) design manner of carrying out the experimental procedure or parameters used therein, or (iii) analyze results or data obtained in the experimental procedures

By yet another aspect there is provided a method for utilizing scientific data, comprising:

(a) compiling scientific data into one or more databases ad loading the databases at one or more addressable sites of a computer network, such that said databases are accessible by users of the network and are arranged in a manner to permit extraction therefrom of experiment-specific data, being data useful for (i) design of experiment tools for a specified experiment, (ii) define procedure or parameters of a specified experiment, or (iii) analyze results or data obtained in an experiment;

(b) permitting users, providing them with means or providing them with instruction for accessing said site and extracting therefrom said experiment-specific data for their specific experiment.

Also provided by the present invention is an experiment kit for use in any of the above methods. Such a kit comprises:

tools for carrying out the experimental procedure; and

means or an instruction set for communicating, over a computer network, with a data-containing site and extracting from said site experiment-specific data employed to (i) define parameters or manner of performing the experiment of procedure or (ii) analyze results or data obtained in the experimental procedure.

By an additional aspect of the invention there is provided a software product carried on a computer readable medium which when loaded into a control processor of an apparatus for carrying out an experimental procedure, can induce the apparatus to communicate, over a computer network, with a data-containing site and extracting from said site experiment-specific data employed to (i) define parameters or manner of performing the experimental procedure, Or (ii) analyze results or data obtained in the experimental procedure. The invention her provides a computer readable medium carrying such a software.

### BRIEF DESCRIPTION OF THE DRAWINGS

In order to understand the invention and to see how it may be carried out in practice, a preferred embodiment will now be described, by way of non-limiting example only, with reference to the accompanying drawings, in which,

FIG. 1A shows a computer network for implementing a method in accordance with an embodiment of the invention, carried out by employment of a processor-comprising apparatus.

FIG. 1B shows a computer network for carrying out a method in accordance with the embodiment of the invention with an apparatus controlled by a computer.

FIG. 2A shows an operational sequence of a method in accordance with an embodiment of the invention.

FIG. 2B shows an operational sequence of the method of the invention in accordance with another embodiment.

FIGS. 3 and 4 show partial sequences of methods in accordance with embodiments of the invention.

### DETAILED DESCRIPTION OF THE INVENTION

In the following, the invention will be explained, at times with specific reference to some specific embodiment including, but not only, those illustrated in the annexed drawings. It should be clear to the artisan that the invention is not limited to the specifically illustrated embodiments the only purpose of which is to illustrate the invention. Use will be made below with the term “apparatus” which is intended to refer both to an apparatus with an integral processor or with an apparatus which cooperates with an external computer which controls its operation.

A computer network which will be referred to specifically is the Internet which is a preferred medium for carrying out the present invention. However, as will be appreciated by the artisan the invention is not limited thereto but rather may also be carried out on a number of different computer networks including internal computer networks such as an intranet, etc.

The term “site” refers to virtual location on a computer network which is characterized by a specific address (R:L in the case of the Internet). As will also no doubt be appreciated, a computer site may at times be hosted on one or more servers of the computer network. As will also be appreciated, a site may at times have different components located on different servers, e.g. a database on one server, analysis of a package on another, etc.

The present invention provides a novel concept in the aft of performing of the experiment. In accordance with the invention utilization of data which can be extracted from a site reachable through a computer network becomes an integral part of the experimental setup. A sequence of communication, extracting data and utilization of such data for the purpose of the experimental procedure becomes like another (virtual) reagent or tool for the performance of the experiment. Such data if properly utilized can be very advantageous to dramatically increase efficiency or accuracy, improve the ability to interpret the results of the experiment, etc. This is achieved as a result of optimizing the conditions or the type of tools to be used for the purpose of a specific experiment, etc.

Communication with a database for the purpose of extracting experiment-specific data may be performed once for each experimental procedure or may be a continuous process. For example, the results of an experimental procedure may be communicated, over the computer network, with the database; based on such result, experiment-specific data may be communicated back to the apparatus for the purpose of either analyzing the result, devising subsequent procedural steps or both.

In general data extracted from the database may be utilized for the control of the parameters employed in design and preparation of said experiment tools, in control of the experimental procedure, or for the purpose of analysis of the results or predicted outcome of the experiment. The term “parameter” includes all the physical and/or chemical parameters employed which may influence the outcome of the experimental procedure. This includes, without limitation, the order of steps of the experimental procedure; the nature of the reagent used in the procedure; the chemical composition of the reaction medium, the temperature; and in general any factor which may influence the outcome of the experimental procedure, the specificity of results sensitively of an assay (where the experimental procedure is a diagnostic assay), the type of experiment end point which may be expected, the speed by which the result or outcome may be obtained, etc.

In accordance with one embodiment the experiment-specific data is used for designing or selecting experiment tools. For example, based on data relating the type of experiment e.g. particulars of the preparation, particulars regarding a certain screening assay, etc., experiment tools such as reagents, substrates for use in the reaction, etc. may be chosen so as to yield optimal results, to make a better interpretation of the results, or the like. A specific example is in designing a DNA chip by including in it an optimal array of probes for specific target sample, tissue or cells.

In accordance with another embodiment, the experiment-specific data are used to define parameters or manner of performing the experiment. This may include selecting reagents, defining the sequence of steps, etc.

At times, in accordance with another embodiment of the invention, the experiment-specific data may be used to predict outcome of subsequent experimental steps. For example, in a polynucleotide amplification experiment, based on the probe and data regarding the target cell, the subsequent elongation steps of the experiment may be predicted. This may considerably shorten the time required for the actual physical reaction and increase its accuracy.

By a still further embodiment, the results of the experimental procedure may be analyzed. For example, in the case of oligonucleotide sequencing operation, sequence data from each step may be compared with sequence data in a database to allow verification of the data, detect instances where stretches of the vector are being sequenced, etc.

The present invention is a novel method and kit. The present invention also provides a software product useful in this method and which may be included in such a kit.

In accordance with a preferred embodiment of the invention, the computer network is the Internet. It should be understood that while, in accordance with one preferred embodiment;, the experimental procedure makes use of an apparatus with the ability to communicate directly, e.g. through a modem, over the computer network with the database-coining site, it is also possible, in accordance with other embodiments of the invention, that the communication over the computer network will be manual, namely by the experimenter. For example, a kit for carrying out an experimental procedure, which comprises various experiment tools may also comprise an instruction set giving, for example, the Internet address (URL) of a site and a certain code which is inputted at the site for the purpose of extracting the experiment-specific data.

In accordance wit one embodiment of the invention the database in said site is a passive database and the auxiliary software accesses the database to extract the relevant experiment-related data, namely data which relates to the particulars of the experimental procedure or of the expected results. The extracted data is then transmitted from the database to the apparatus for processing there. Alternatively, the auxiliary software may interact with a database-associated software, e.g. an expert software system, and this database-associated software then interacts with said auxiliary software to transmit a relevant experiment-related data accompanied at times by some software applications which then runs on said processor for control of manner of the parameters employed in the experimental procedure, or for analysis of the results or outcome of the experiment.

The parameters of the experimental procedure, which may be influenced by the extracted data may include

(i) The sequence of steps carried out in the experimental procedure. This may include the order of steps, omission or addition of steps, etc.

(ii) Physical or chemical parameters applied during the experimental procedure, This may include, for example, temperature, chemical composition of the medium in which the biological or biochemical reaction of the experimental procedure takes place, the temperature, concentration of ingredients or reagents employed in the experimental procedure, etc,

(iii) The type of reagents which are utilized in the experimental procedure. This may include the nature of the diagnostic entities, type of substrates used for the reaction (e.g. solid states, substrates, such as beads or chips carrying molecular entities used in the experimental procedure, etc.), and others.

(iv) The type of experimental end point. Very often, an experimental procedure may have a different kind of measurable effects, for example, temperature, concentration of a reagent or an analyte, measurement of a detectable label (which may be selected out of a variety of such labels), etc. It is often critical to choose the correct end point in order to allow determining of a certain biological/biochemical effect. At times it is important not only to choose a type of end point but also to determine how it should be arrived at. For examples in the case of a quantitative PCR, the determination may be of the number of cycles and/or temperature employed needed in order to obtain a quantitative measurement (and to avoid reaching saturation levels on the one hand and low signal-to-noise ratio results on the other hand.)

An experimental procedure performed with some knowledge as to a potential outcome or result may be optimized to allow to achieve maximal or easily measurable effect. Thus, in accordance with one embodiment of the invention, the data extracted from the database is employed in order to predict the results or outcome of the experimental procedure and the parameters of the experimental procedure may then be modified based thereon. For example, in the case of an assay intended to determine the level of expression of a certain expressed mRNA, a data on expected level of expression, may allow to control the parameters of the experimental procedure such that the measured end effect will linearly depend on the level of the target sequence. In addition, in the case of a certain end point expected in view of data extracted from the database, the experimental procedure may be modified to more rapidly achieve such an end point. For example, where a certain oligonucleotide is sequenced, based on homology with other sequences, a prediction can be made as to the next base to be determined (whether it is A, T, C or G) and then the search for the next base may be geared towards detection of the expected next base. Occasionally, this may be inaccurate (in case of 90% identity, one out of ten predicted bases will be incorrect) but overall the speed by which a sequencing procedure may be carried oat will be much faster. In the case of sequencing, communication with the database may be ongoing, e.g. following each sequencing step.

In the case of an experimental procedure aimed in detection of expressed sequences in a sample, e,g. in order to determine differences in expression pattern between two biological samples, a chip may be used having certain desired probes carried thereon. The choice of probes may obviously be detrimental as to the ability to discern differences between two samples, e.g. one normal sample and one obtained from a diseased tissue. In accordance with an embodiment of the invention, a database including data regarding level of expression of different sequences may be used in order to first carry out a “virtual experiment” and determine whether based on such a virtual experiment, differences between the different types of tissue may be discerned. In addition to expression levels, data regarding “noise” namely background detection levels of the different sequences may also be factored. If the chip is not suitable for the intended purpose, the user may be prompted to use a different DNA chip for the purpose.

The invention permits as use of data extracted from the database for the purpose of analyzing results. In accordance with an embodiment of the invention, for the purpose of analysis of results or outcome of experimental procedure, the extracted data is processed in combination with the experiment data. In this manner, one or more of the following may be achieved:

(i) Potential interpretations of the results or outcome of an experimental procedure may be achieved. Such data may be required in order to determine, on the basis of the measured results, the type of analytes which existed in a tested sample. For example, a pattern of signals detected an a DNA chip may be analyzed to determine the nature of the expressed nucleotide sequences in the assayed sample. By another example, a mass-spectra pattern may be compared to a database in order to determine the type of molecules in the sample. By a fiber example, a chromatography, e.g. a thin layer chromatography (TLC) pattern, which may be one or two dimensional, may be analyzed, based on a database on migratory patterns of different molecules under different conditions, to determine the type of molecules found in a sample.

(ii) The extracted data may be employed in order to provide one or more potential interpretations of biological significance of the obtained result or outcome. For example, an expression pattern as detected on a DNA chip, may be used, in connection with data relating to level of expression of different polynucleotide sequences in different cells and/or under different conditions; for determining the nature of the sample and/or whether it is a normal sample or whether it is diseased. By another example, the type of molecules, including without limitation the type of nucleotide sequences detected in the sample, the existence of a certain pathogen may be determined.

(iii) In a multi-step experimental procedure, results of one step may be used in order to predict outcome of a subsequent step of the experimental procedure. For example, in a sequencing experimental procedure, homologous sequences to a certain sequenced stretch of unknown polynucleotide, may be used in order to predict a subsequent polynucleotide and the entire experimental setup may be changed to be geared towards detection of the expected polynucleotide, Such procedure may considerably shorten the time required for a sequencing operation and increase its accuracy. Furthermore, at times, in the sequencing operation, portions of the vector may be mistakenly sequenced as well. By use of a database on vectors to sequences and extraction of such data immediately when a nucleotide stretch of the vector is being sequenced, an alert may be initiated and the sequencing operation stopped to avoid sequencing of such non-coding sequence.

In accordance with one preferred embodiment of the invention, the experimental procedure comprises deciphering of the sequence of one or more polynucleotides or reacting one or more first polynucleotides with one or more second polynucleotides. The deciphering sequences of polynucleotides is routinely performed by the use of high throughput screening robotic systems which automatically decipher sequences of polynucleotides of interest, Reacting polynucleotides with other polynucleotides may be in the case of a diagnostic assay, e.g. detecting the presence of target oligonucleotides in a sample using one or more probe oligonucleotides immobilized on a substrate. Another example of reacting oligonucleotides with other oligonucleotides is in the case of PCR (polymerize chain reaction), particularly quantitative PCR In accordance with this embodiment the database may comprise one or more of the following:

(i) A sequence data, namely data on full sequences of polynucleotides. In the case of a partial sequence, such data, extracted after obtaining a partial sequence result, may be used in order to predict a more complete length polynucleotide.

(ii) Data on presumed unknown biological significance or expression data of sequences. This includes information on expression of RNA in various types of tissues or cells or pattern of expression under different conditions, e.g. in a normal or in a pathological state, etc.

(iii) Data and homologs. Such data may be used, as already pointed out above, in order to predict nonciphered sequence particulars of a partially sequenced polynucleotide.

(iv) Data on splice variants. Splice variants data may be highly important both for sequencing operations as well as for diagnostic assays. For example, different splice variants may bind to the same probe and information on, for example, expression of a specific splice variant in a specific tissue may be important in order to interpret result obtained, for example, by analyzing the binding to a DNA chip. In accordance with the invention, data on splice variants extracted from the database may be used to predict expected binding of different splice variants onto a specific DNA chip and potentially choose another DNA chip if the splice variant binding may mask the date of interest. In addition, by processing data of splice variants, there may be a better interpretation of results obtains in a specific tissue, The sequence database may typically be obtained through analysis of EST data.

In addition to the application of the invention for experimental procedures involving polynucleotides, the invention is also applicable in experimental procedures which comprise reacting, detecting or analyzing one or more biological molecules other than oligonucleotides. The relevant database in such a case would include data relevant to such biological molecules. Such data may include chemical or physical characteristics, namely empirical formulae, mass-spectra pattern hydrophobicity, solubility in different solutions, molecular weight, chemical structure, reactivity with different reagents, etc. Furthermore, such a database may also include data on biological function or biological significance, as for example the biological role of a specific molecule, correlation between its level and a certain diseased state, et. Additionally, the data on such molecules may also include a distribution pattern in different tissues as well as a type of expected interactions of the molecules with other molecules. Such biological molecules may be one or more of the group consisting of polypeptides Or proteins, carbohydrates, lipids and a variety of small molecules (small molecules are molecules of a molecular weight typically less than 1,000 Daltons).

In accordance with one embodiment of the invention the experimental procedure is a multi-step process and each step is dependent on results or outcome of a previous step. In accordance with this embodiment the extracted data may be processed to ether refine conditions or predict results or outcome of one or more subsequent experimental steps, in a manner as described above.

The system of the invention will typically comprise at least one server linked to a Computer network, typically the Internet while the apparatus or a computer associated therewith has a communication port permitting it to communicate with the server through the network.

### DESCRIPTION OF SPECIFIC EMBODIMENTS

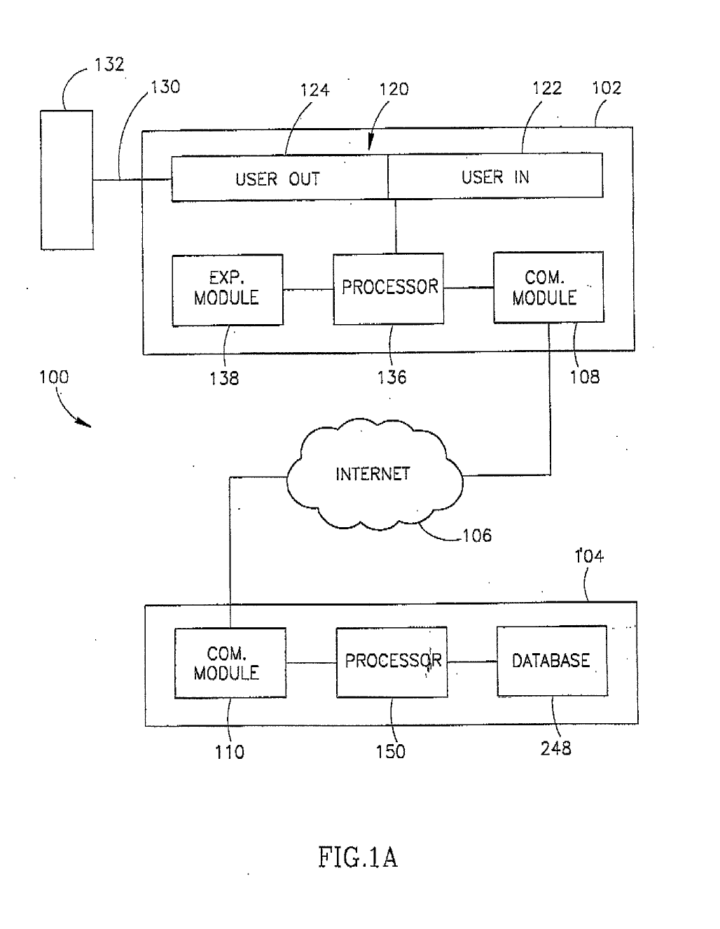

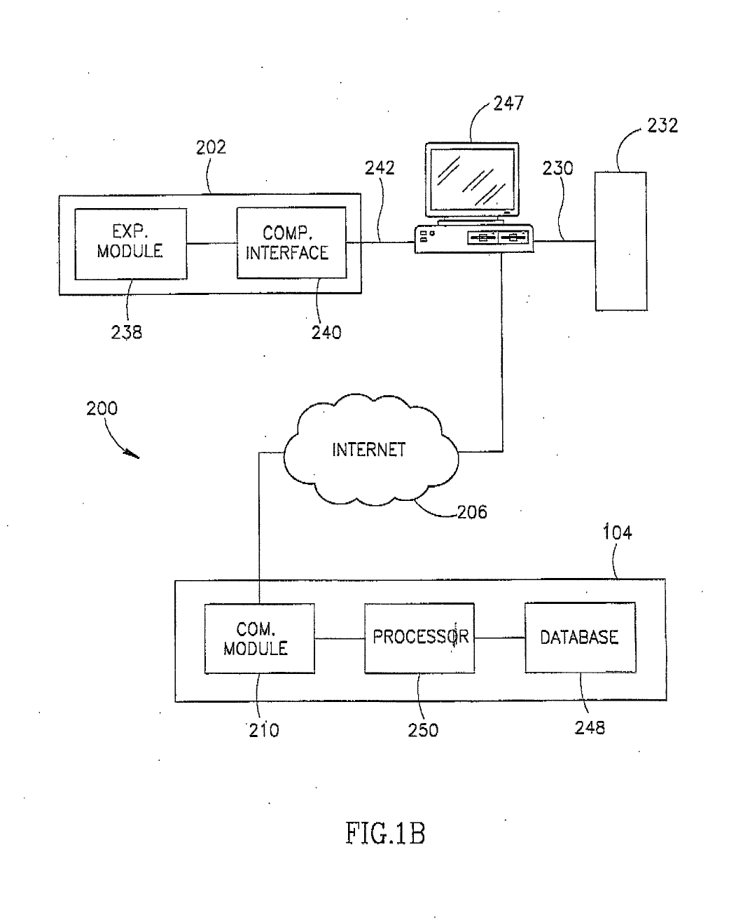

Reference is now being made to FIGS. 1A and 1B showing computer networks 100 and 200, respectively, in accordance with the embodiments of the invention. In FIGS. 1B, like components to those of FIG. 1A having given the same numeral shifted by 100 (e.g. component 104 in FIG. 1A is identical to component 204 in FIG. 1B). Network 100 shown in FIG. 1A comprises an apparatus 102 and a server 104 both linked to the Internet 106 through respective communication modules 108 and 110. Apparatus 102 comprises a user interface generally designated 120 consisting of one module 122 permitting the user to input various parameters relating to the experimental procedure and another module 124 for outputting the outcome or result of the experimental procedure. This outcome may be displayed to the individual. In addition, this outcome may be communicated through the link 130 to an auxiliary computer 132 for storage and analyzing the output.

Apparatus 102 comprises also a processor 136 and an experiment module 138. The experiment module is a module containing all the necessary units, devices, probes, reactor flow systems, etc. needed in order to carry out the intended experimental procedure. For example, in the case of a diagnostic assay this includes means to carry out the necessary diagnostic reaction, In accordance with another example, in the case of a high throughput screening assay, the experimental module may comprise a robotic system for automatic carrying out the necessary experimental step. Generally, the experimental module can be any such module existing in apparatuses for carrying out the biological experimental procedures, and known per se.

The network 200 in FIG. 1B differs from that in FIG. 1A in that the apparatus 202, having an experimental module 238, comprises a computer interface 240 linked through communication line 242 to a computer 247. Computer 247 in this case serves both as a user interface as well as for control of apparatus 202. Computer 247 is linked through a communication port (not shown) to the Internet.

Servers 104 and 204 also holds a database 248 and an associated processor 150. Database 248 is accessible by the auxiliary software ran on processor 136 or computer 247 to extract therefrom relevant data. A database-associated software may may on processor 150, 250 of server 104, 204 for interaction with the auxiliary software.

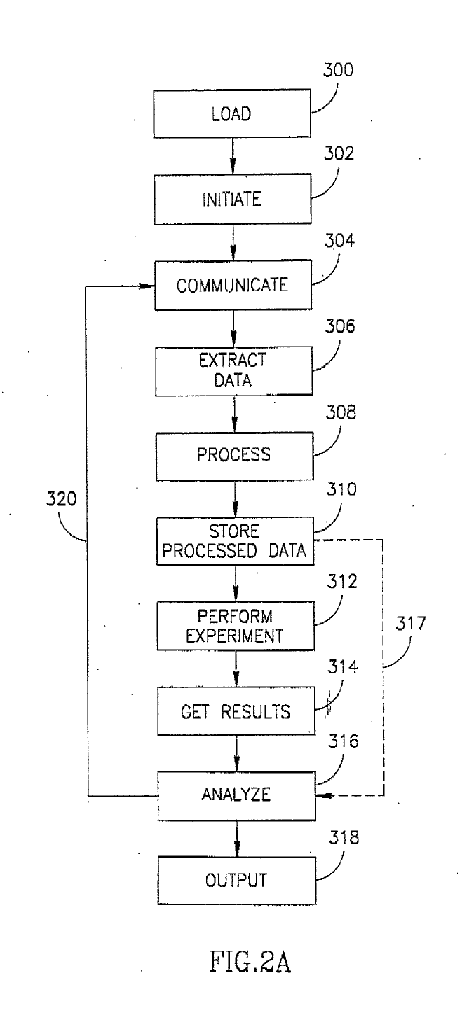

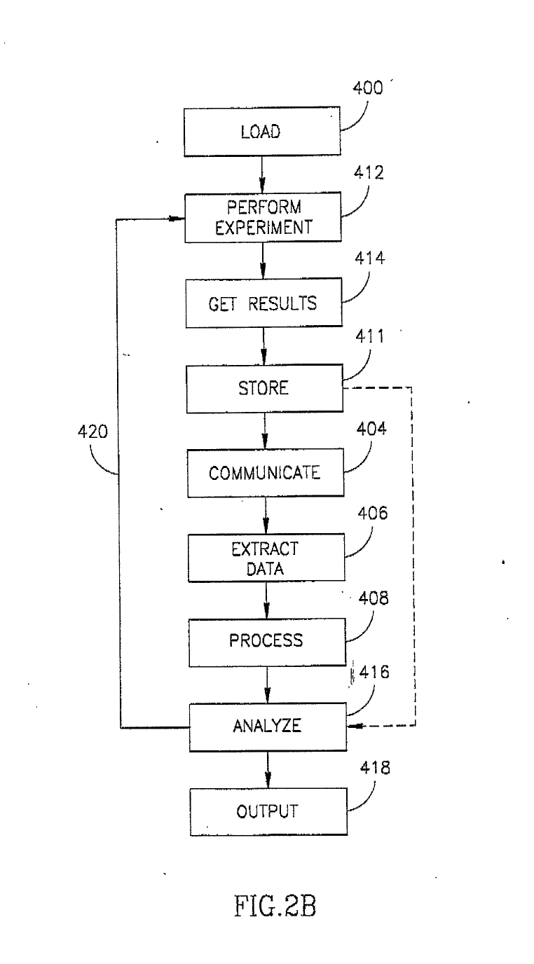

Shown in each of FIGS. 1A and 1B is one server (104 and 204, respectively). As will be appreciated, this is an example only and such a network may at times include additional servers of the invention, each being a mirror of one another, each containing a portion of the database, one containing a database and one processing software, etc. Such one or more servers constitute a “site” as defined above, Reference is now being made to FIG. 2A showing an operational sequence in accordance with the invention. In a first step (300) the auxiliary software is loaded into the control processor of the apparatus or into a computer associated with the apparatus. Upon initiation of the start of an experimental procedure (302), the auxiliary software loaded (300) first induces the apparatus or a computer associated therewith to communicate (304) with a server containing a database with data relevant to the experiment and data is extracted (306), processed (308) and then the processed data is stored (310) in a manner allowing its subsequent use for analysis. Then the experiment of procedure is carried out (312) and the obtained results (314) are then analyzed (316) together with the forwarded (317) stored process data. Results may then be outputted (318) or alternatively, where the experimental procedure is a multi-step process, the analyzed results may then be fed as input for a subsequent step (marked by return arrow 320),

The sequence of operation for carrying out an experimental procedure in accordance with another embodiment of the invention is shown in FIG. 2B. In FIG. 2B, similar steps are marked by the same reference numerals, shifted by 100, as the corresponding reference numerals in FIG. 2A, The operational sequence shown in FIG. 2B differs from FIG. 2A in that rather than etracting data and then carrying out the experimental procedure, in this case the experiment is performed first, results are stored (411) and eventually the stored results are analyzed in combination with extracted and processed (406, 408) data. Similarly as in FIG. 2A, the analyzed data may be fed back (420) for performance of a subsequent step, in the case of a multi-step process.

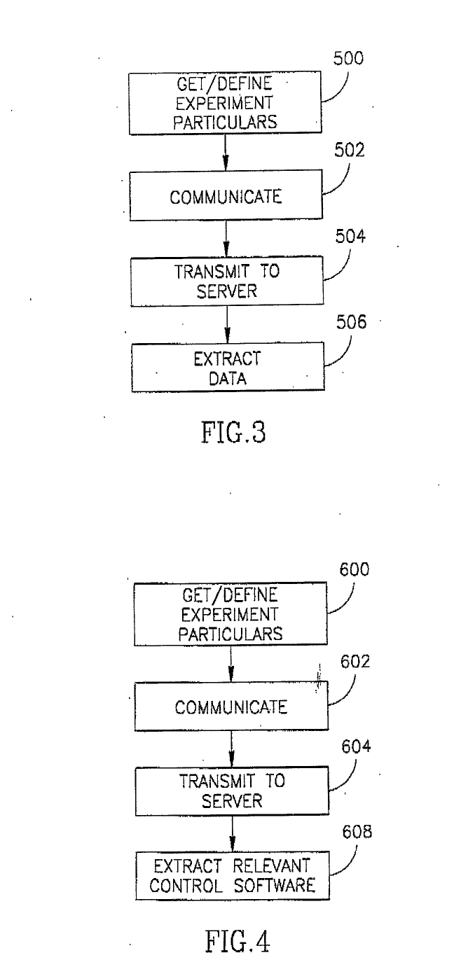

Reference is now being made to FIGS. 3 and 4 showing two modes of the manner of communication between the apparatus and the server. In a first step 500 the auxiliary software obtains experiment particulars which may be inputted by the user, defined automatically within the apparatus or transmitted to the apparatus from an external source, e.g. from another computer. The server is communicated (502) and data relating to the experiment particulars are then transmitted to the server (504). In a subsequent step (506) the server transmits the relevant data back to the apparatus.

FIG. 4 shows a similar sequence and here again, the same steps are given with the same reference numeral shifted by 100. The difference in FIG. 4 is that rather than a simple data, extracted is also relevant control software subsequently operating within the apparatus either in controlling the manner of performing the experimental procedure or the manner in which results are analyzed,

### EXAMPLES

Sequencing in Conjunction with Bioinformatics

Today's nucleotide sequencing reactions are carried out by automated machines, designed to identify the length of nucleotide fragments and the wavelength emitted by their dideoxy terminators, based on the Sanger sequencing model. Eventually, the laser trace files obtained from these reactions are submitted to a separate module, which determines the nucleotide sequence based on the relative emission of the different wavelength. This procedure does not rely on any external sequence information in order to provide results. For instance, it may not detect incorrect or redundant sequences, and it cannot create any feedback to the sequencing machine itself

In accordance with the invention, the technology of the sequencing machine is combined with a sequencing database, the two being connectible through a computer network. By analyzing the output sequence as it is created, based on data received from the database, the results may be compared with existing databases, and provide the following alerts and decisions:

(i) Detection and cleaning of vector sequences. In specific cases, entry into a vector sequence shall either cause an alert or halt the sequencing process altogether, thus freeing the machine for a new reaction.

(ii) Detection of chimeric clones: by comparing the output with existing databases, the systems will be able to alert the user once non-expected sequence is created, alerting him to possible concatenation or chimeric regions in the clone.

(iii) Nucleotide determination: when the base-calling software is not confident of the right nucleotide in a certain position, the system may help it by finding the expected letter using sequences previously obtained.

(iv) Polymorphic area: the system indicates possible SAPs and other polymorphic areas, such as CA-repeats and other microsatellites, during the progress of the sequencing process.

(v) when a largescale sequencing project takes place, using the data obtained up to this point may help identify redundancy in the clone analyzed, and automatically eliminate overlaps where the sequence is already finalized.

(vi) Diagnostics and SAP: by identifying a mutation or a polymorphic site in diagnostic samples, the system may alert the user even before process is complete Then, the system controls a decision tree, in which other sites are selected for amplification, sequencing, or targeted site analysis according to the preliminary results, making human intervention redundant.

In all the above cases, the integration of the bioinformatics database with the apparatus allows a control of the apparatuses operation, by halting the apparatus when non-relevant sequence is reached, reloading the same sample for validation, and providing various advisory messages.

Bioinformaties—enhanced Quantitative PCR

The use of RT-PCR as an expression level measurement tool has been impeded by the exponential nature of the amplification, which rapidly becomes insensitive to the RNA template starting amount. In order to solve this problem, linear, rather than exponential, RT-PCR has been attempted, Still, the variations in the template starting quantity, in several orders of magnitudes, make it difficult to calibrate a specific reaction so the obtained DNA amount will enter detectable levels without reaching saturation (where the exact starting quantity loses its importance).

In accordance with the invention, data relating to expression of specific sequences m various target tissues, communicated through a computer network, permits better calibration of the PCR, both in terms of reaction temperature and the number of cycles. Expected data on genes and their expression patterns allow to calibrate the PCR to remain in the linear amplification phase for each specific gene-tissue combination.

Instant RACE

Marathon-RACE is a biological procedure for the extension of known, expressed sequences, in the form of cDNA, into full length RNA transcripts. In the typical case, a RACE experiment to extend a few hundred base-pairs to a 3-4 kilobase RNA takes anywhere tour a few weeks to a few months and a considerable amount of molecular biology reagents.

In accordance with the invention data on sequences is used to shorten the RACE procedure. The RACE procedure is begun by running an extension analysis, and by integrating data from a sequence database, elongation information to the starting sequence may virtually be added. Furthermore, based on the chosen primers, prediction may be made on which splice variants are expected to emerge from the RACE amplification process, to create a simulation of the transcript picture output, prior to performance of the experimental procedure.

Chip Design Using Sequence and Expression Information

The use of DNA chips has become abundant in both research and diagnostics settings, as the attempt to discover differences in genomic composition or RNA expression level is accelerated for the purpose of better understanding disease and treatment possibilities.

The nature of a DNA chip experiment consist of the positioning of a multitude of probes in very close proximity to each other, exposing the chip to the target DNA/RNA solution, then scanning and analysing the differences in emittance intensity derived from local hybridisation levels.

The problem of obtaining a significant signal-to-noise ratio dictates the use of multiple probes per gene. As the information on most genes is still partial and based on EST sequences, it is sometimes hard to define when two seemingly foreign probes actually belong to the same gene. Moreover, when two probes are derived from different splice variants of the same gene, they will be identified as belonging to the same gene. Another problem is that of the ‘spillover’ effect—a probe A adjacent to a ‘full hit’ probe B might be ‘lit’ and indicated positive if a the target gene bears some resemblance to probe A in a different section of its sequence.

In accordance with the present invention a DNA arrayer or a similar system, which is used to create DNA chips, may be combined, in accordance with one embodiment (“Embodiment A”) with gene and splice variant formation, through an automated network system. In accordance with another embodiment (“Embodiment B”) a DNAchip scanner may be combined with milar information. In both cases the information might include both sequence and expression data, derived from various sources such as differential display or SAGE libraries

Embodiment A: Normally, the chip probe composition and mapping is designed separately, and the arrayer receives only precise dispensing instructions. In the embedded system, however, the arrayer will use the probe and chip map information for further quality control, Using the data available from the network, the arrayer will detect same-gene probes, splice variants, and potential spillover sites, and either alert the user or propose alternative design for his approval.

Using expression information, such as SAGE, the system will simulate the chip assay and predict the array hybridisation results. Again, this information will be used to optimize chip design, for example by keeping high-intensity spots separated enough to avoid local signal saturation.

Embodiment B: The analysis of a complex pattern is facilitated by providing the system some preliminary prediction on the pattern. Using chip design information, coupled with SAGE expression data and other sources, including previous experiment's results, the system simulates the result pattern of the experiment and provides it back through the network to the scanner as reference. This simulation is helpful in calibrating the scanner sensitivity and gain to the overall signal expected from this specific chip. Moreover, instead of using two DNA chips for each differential expression analysis and comparing their results, this embodiment creates expression assays using only one real chip and one ‘virtal’ chip, where the base results for the experiment are already known, Thus the scanning system is able to highlight unexpected results and new differentially expressed genes.

1. A method for performing an experiment comprising
(a) providing or selecting one or more experiment tools,
(b) performing said experiment utilizing said tools, and
(c) analyzing results;
said method being characterized in that it comprises communicating through a computer network with a data-containing site of the network to extract therefrom experiment-specific data and utilizing said data for either one or more of (i) designing or selecting said tools, (ii) defining parameters or manner of performing the experiment, (iii) predicting outcome of one or more subsequent steps of the experiment and (iv) analyzing the results or data obtained in the experiment.

2. A method according to claim 1, wherein the computer network is the Internet.

3. A method according to claim 1, wherein at least one of steps (a), (b) or (c) is performed by an apparatus that comprises a processor loaded with a software for communicating with said site and for extracting and processing said data.

4. A method according to claim 1, wherein said experiment is a biological or biochemical experimental procedures and said site holds biological data.

5. A method according to claim 4, wherein said experimental procedure is intended to analyze or utilize biological macromolecules and said data is data on composition, chemical properties, physical properties, biological properties or function, distribution thereof in different biological tissues and data on closely related macromolecules.

6. A method according to claim 5, wherein said macromolecules are nucleic acid or polypeptide.

7. A method according to claim 6, wherein said experiment comprises one of the following:
sequencing a nucleic acid sequence with said data being used to either predict outcome of one or more subsequent sequencing steps, verity sequencing data of a previous one or more sequencing steps, or both;
amplifying specific sequences in a sample with said data being used to select a primer or conditions for the amplification reaction;
extension of expressed partial sequences into longer stretches of expressed sequences; and
selecting one or more probes for detecting one or more nucleic acid sequences in a sample.

8. A method for assembling experiment tools for use in an experiment, comprising annexing to or associating with said tools means or an instruction set for communicating, over a computer network with a data-containing site and extracting from said site experiment-specific data, the extracted data being employed to (i) define parameters or manner of performing the experiment, or (ii) analyze results or data obtained in the experiment.

9. A method according to claim 8, wherein said means comprise a computer readable medium carrying software loadable into a computer or into a processor of an apparatus that executes the experiment, whereby said computer or apparatus can communicate with said site, extract said experiment-specific data and process it for use in said experiment.

10. A method according to claim 8, wherein said data is a biological or a biochemical data and said experiment is a biological or biochemical experiment.

11. A method according to claim 10, wherein said experiment is intended to analyze or utilize biological macromolecules and said data is data on composition, chemical properties, physical properties, biological properties or function, distribution thereof in different biological tissues, or data on closely related macromolecules.

12. A method according to claim 11, wherein said experiment is intended to screen for a plurality of different macromolecules in a biological sample.

13. A method according to claim 11, wherein said macromolecules are polynucleotides or polypeptides.

14. A method according to claim 13, wherein said macromolecules are polynucleotides and said site holds polynucleotide sequence data and data on tissue expression pattern of polynucleotides.

15. A method according to claim 14, wherein said experiment comprises one of the following:
sequencing a nucleic acid sequence with said data being used to either predict outcome of one or more subsequent sequencing steps, verity sequencing data of a previous one or more sequencing steps, or both;
amplifying specific sequences in a sample with said data being used to select a primer or conditions for the amplification reaction;
extension of expressed partial sequences into longer stretches of expressed sequences; and
selecting one or more probes for detecting one or more nucleic acid sequences in a sample.

16. A method according to claim 8, wherein said tools and said means are assembled together into an experiment kit.

17. A method comprising:
(a) collecting scientific data
(b) assembling said data into a database in a manner permitting extraction therefrom of experiment-specific data and utilization of said data for either (i) design of tools for carrying out a specified experiment, (ii) define parameters for carrying out a specified experiment or (in) analyze as results or data obtained in an experiment;
(c) loading the database onto a site of a computer network in a manner permitting users of the network to communicate therewith and to extract therefrom the experiment-specific data for utilizing the extracted data in their experiment in a manner as defined in step (b).

18. A method according to claim 17, comprising the following additional step:
(d) providing a user who intends to perform an experiment with software or au instruction set, for communicating with and extracting said data from said site.

19. A method according to claim 18, wherein said software processes said data to permit its utilization for said experiment.

20. A method for assisting an experiment performer in performing an experimental procedure, comprising the steps of:
(a) providing a user accessible site on a computer network in a manner permitting users of the network to communicate and exchange data therewith, the site holding a database of data and software permitting to search the database and extract experiment-specific data therefrom, said data being data that may be used by the experimenter in designing an experimental procedure or analyze results or data obtained in such a procedure; and
(b) permitting the experimenter to communicate with said site through the computer network and in response to data transmitted from the experimenter to said site defining particulars of the experiment, transmitting to the experimenter said experiment-specific data or said data in combination with software, for (i) designing or selecting experiment tools for carrying out the experimental procedure, (ii) design manner of carrying out the experimental procedure or parameters used therein, or (iii) analyze results or data obtained in the experimental procedure.

21. A method according to claim 20, wherein sad network is the Internet.

22. A method according to claim 20, comprising updating data in said database.

23. A method according to claim 20, wherein said experiment performer is an apparatus which is either (i) an apparatus that prepares or assembles experiment tools for carrying out the experimental procedure, or (ii) an apparatus in which the experimental procedure is carried out or in which the results or data obtained or collected in said experimental procedure are analyzed; the apparatus being connected or connectable to the computer network and is capable of communicating automatically with said site to extract the needed data.

24. A method according to claim 23, wherein said apparatus has a processor loaded with software for such communication and data extraction.

25. A method according to any one of claims 20, comprising the following step (aa) between steps (a) and (b);
(aa) providing the exerimenter with at least one of:
software for exchange of data with said site;
an instruction set for communicating and exchanging data with said site;
an accession code for accessing said data base; and
a specified address within said site for downloading experiment-specific data.

26. A method according to claim 25, wherein said software is annexed to or associated with experiment tools provided for performance of the experimental procedure.

27. A method according to claim 26, wherein said tools and said software are provided together as a kit.

28. A method according to claim 27, wherein said kit is provided together with instructions for use, said instructions being in a written form or being embedded in said software.

29. A method according to claim 28, wherein said instructions are displayed on a display of said apparatus or on a display associated therewith after loading of said software.

30. A method according to claim 20, wherein said experimental procedure is a biological or a biochemical experimental procedure.

31. A method according to claim 30, wherein said experimental procedure is intended to analyze or utilize biological macromolecules and said data is data on composition, chemical properties, physical properties, biological properties or function, distribution thereof in different biological tissues, or data on closely related macromolecules.

32. A method according to claim 31, wherein said experiment is intended to screen for a plurality of different macromolecules in a biological sample.

33. A method according to claim 31, wherein said macromolecules are polynucleotides or polypeptides.

34. A method according to claim 33, wherein said macromolecules are polynucleotides and said site holds polynucleotide sequence data and data on tissue expression pattern of polynucleotides.

35. A method according to claim 34, wherein said experiment comprises one of the following:
sequencing a nucleic acid sequence with said data being used to either predict outcome of one or more subsequent sequencing steps, verity sequencing data of a previous one or more sequencing steps, or both;
amplifying specific sequences in a sample with said data being used to select a primer or conditions for the amplification reaction;
extension of expressed partial sequences into longer stretches of expressed sequences; and
selecting one or more probes for detecting one or more nucleic acid sequences in a sample.

36. A method for utilizing scientific data, comprising:
(a) compiling scientific data into one or more databases and loading the databases at one or more addressable sites of a computer network, such that said databases are accessible by users of the network and are arranged in a manner to permit extraction therefrom of experiment-specific data, being data useful for (i) design of experiment tools for a specified experiment, (ii) define procedure or parameters of a specified experiment, or (iii) analyze results or data obtained in an experiment;
(b) permitting users, providing them with means or providing them with instruction for accessing said site and extracting therefrom said experiment-specific data for their specific experiment.

37. A method according to claim 36, wherein said network is the Internet.

38. A method according to claim 36, wherein the collected scientific data is data on composition, chemical properties, physical properties, biological properties or function, distribution thereof in different biological tissues, or data on closely related macromolecules.

39. A method according to claim 38, wherein the collected scientific data is data on sequences of nucleic acids or on expression of nucleic acid sequences in different cells or tissue.

40. A method according to claim 39, wherein the scientific experiment is an experiment in which a nucleic sequence is sequenced or used as a probe or a reagent.

41. A method according to claim 40, wherein said experiment comprises one of the following:
sequencing a nucleic acid sequence with said data being used to either predict outcome of one or more subsequent sequencing steps, verify sequencing data of a previous one or more sequencing steps, or both;
amplifying specific sequences in a sample with said data being used to select a primer or conditions for the amplification reaction;
extension of expressed partial sequences into longer stretches of expressed sequences; and
selecting one or more probes for detecting one or more nucleic acid sequences in a sample.

42. A kit for use in an experimental procedure, comprising:
tools for carrying out the experimental procedure; and
means or an instruction set for communicating, over a computer network, with a data-containing site and extracting from said site experiment-specific data employed to (i) define parameters or manner of performing the experimental procedure, or (ii) analyze results or data obtained in the experimental procedure.

43. A kit according to claim 42, wherein said tools comprise one or more of reagents, disposables and devices.

44. A kit according to claim 42, comprising a computer readable medium carrying software loadable into a computer or into a processor of an apparatus that executes the experiment, whereby said computer or apparatus can communicate with said site, extract said experiment-specific data and process it for use in said experiment.

45. A kit according to claim 42, for use in a biological or biochemical experimental procedure.

46. A kit according to claim 44, for use in an experimental procedure in which biological macromolecules are analyzed or utilized and said means or an instruction set are for communicating with a database and extracting data therefrom on composition, chemical properties, physical properties, biological properties or function, distribution of the macromolecules in different biological tissues, or data on closely related macromolecules.

47. A kit according to claim 45, wherein the biological macromolecules are polynucleotides or polypeptides.

48. A kit according to claim 47, for use in a experimental procedure selected from the group consisting of:
sequencing a nucleic acid sequence with said data being used to either predict outcome of one or more subsequent sequencing steps, verify sequencing data of a previous one or more sequencing steps, or both;
amplifying specific sequences in a sample with said data being used to select a primer or conditions for the amplification reaction;
extension of expressed partial sequences into longer stretches of expressed sequences; and
selecting one or more probes for detecting one or more nucleic acid sequences in a sample.

49. A kit according to claim 48, comprising polynucleotide detection chip.

50. A kit according to claim 48, comprising;
tools for carrying out a polynucleotide amplification or a partial expressible-sequence extension reaction; and
software or an instruction set for communicating, over a computer network with a site holding a database for extracting data useful for defining parameters for carrying out the experimental procedure, analyze data or result of the experimental procedure, or predict outcome of at least one step of the experimental procedure.

51. A software product carried on a computer readable medium, which when loaded into a control processor of an apparatus for carrying out an experimental procedure, can induce the apparatus to communicate, over a computer network, with a data-containing site and extracting from said site experiment-specific data employed to (i) define parameters or manner of performing the experimental is procedure, or (ii) analyze results or data obtained in the experimental procedure.

52. A computer readable medium carrying a software product which can be loaded into a control processor of an apparatus for carrying out a biological or biochemical experimental procedure, and which once loaded into said control processor can induce the apparatus to communicate, over a computer network, with a data-containing site and extract from said site experiment-specific data employed to (i) define parameters or manner of performing the experimental procedure or (ii) analyze data obtained in the experimental procedure.
